(19) United States
(12) Patent Application Publication
(10) Pub. No.: US 2014/0349345 A1
(43) Pub. Date: Nov. 27, 2014

(54) CYCLOPEPTIDE FERMENTATION AT INCREASED METAL ION CONCENTRATION

(71) Applicant: DSM Sinochem Pharmaceuticals Netherlands B.V., Delft (NL)

(72) Inventors: Wouter Adrianus Van Winden, Echt (NL); Rudolf Van Der Pol, Echt (NL)

(21) Appl. No.: 14/371,789

(22) PCT Filed: Jan. 7, 2013
(86) PCT No.: PCT/EP2013/050142

§ 371 Date: Jul. 11, 2014

(30) Foreign Application Priority Data
Jan. 13, 2012 (EP) 12151025.9

(51) Int. Cl. C07K 7/64; C12R 1/645; C12P 21/02

(52) U.S. Cl. CPC C07K 7/64; C12P 21/02; C12R 1/645
USPC 435/71.1; 435/255.1

(57) ABSTRACT

The present invention relates to a method for the fermentation of cyclopeptides such as pneumocandins in the presence of increased concentrations of any or all of calcium, copper, iron, magnesium, manganese, molybdenum and zinc ions.

### FIELD OF THE INVENTION

The present invention relates to a method for the fermentation of cyclopeptides such as pneumocandins in the presence of increased concentrations of any or all of calcium, copper, iron, magnesium, manganese, molybdenum and zinc ions.

### BACKGROUND OF THE INVENTION

Cyclopeptides are polypeptides in which the terminal amine and carboxyl groups form an internal peptide bond. Hence, cyclopeptides do not have a terminal amine or carboxyl group, although non-terminal amine and carboxyl groups may be present stemming from individual amino acids such as aspartic acid, glutamic acid, lysine and the like. Several cyclopeptides are known for their advantageous medicinal properties. An excellent example is the class of echinocandins which are potent antifungals. Cyclopeptides can be naturally occurring compounds but may also be obtained by total synthesis or by synthetic or enzymatic modification of naturally occurring or naturally produced precursors; the latter class is referred to as semi synthetic cyclopeptides. Examples of medicinally useful echinocandins are the cyclic hexapeptides anidulafungin, caspofungin, cilofungin and micafungin which are useful in treating fungal infections especially those caused by Aspergillus, Blastomyces, Candida, Coccidioides and Histoplasma. These cyclic hexapeptides are characterized in that they comprise threonine and a proline derivative such as 3-hydroxyproline, 4-hydroxyproline and/or 3-hydroxy-4-methylproline. Anidulafungin, caspofungin and micafungin are all semi synthetic cyclopeptides derivable from naturally occurring echinocandins such as for instance echinocandin B, pneumocandin A₀ or pneumocandin B₀.

Pneumocandin B₀ (1, with R₁=C(O)(CH₂)₈CH(CH₃)CH₂CH(CH₃)CH₂CH₃), first disclosed in U.S. Pat. No. 5,202,309, is a compound that can be obtained fermentatively, for instance in Glarea lozoyensis as described in WO 00/08197. The compound is an important intermediate in the preparation of therapeutically active semi synthetic cyclopeptides such as caspofungin, as described in WO 2010/128096 and references cited therein.

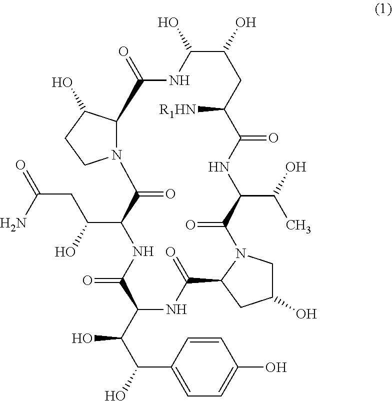

Although nature can provide a substantive part of the complex chemical structure of semi synthetic cyclopeptides, and in many cases having all chiral centers in the required configuration, a major disadvantage nevertheless is that during fermentation often side products are formed that carry through the process and eventually end up as impurities. Only in few cases and after extensive research resulting in unpredictable breakthroughs, can fermentation processes be tuned in such a way as to prevent formation of these impurities. Particularly when these impurities are structurally closely related to the main product, their removal is usually tedious and often requires unprecedented purification approaches as the main products in question are chemically unstable and/or prone to racemization.

In the case of pneumocandin B₀ a multitude of structurally related impurities occurring during fermentation has been described. Examples are compounds having an additional methyl function (such as pneumocandin A₀, pneumocandin A₁, pneumocandin A₂, pneumocandin A₃, pneumocandin A₄, pneumocandin A₅, pneumocandin A₆), compounds lacking one or two hydroxyl groups (such as pneumocandin B₁, pneumocandin B₂, pneumocandin B₅, pneumocandin B₆, pneumocandin E₀), compounds having a 4-hydroxy proline rather than a 3-hydroxy proline moiety (pneumocandin C₀) or compounds having additional hydroxyl groups (such as pneumocandin D₀, pneumocandin D₂).

Improvement of the fermentation process is the subject of WO 00/08197, which document addresses increase in the production titer of pneumocandin B₀, but also reduction of the formation of unwanted structurally related impurities. Notably favorable results where achieved by supplementing the amino acids arginine, glutamine, hydroxyproline, ornithine, proline or threonine and the trace elements boron, calcium, cobalt, copper, iron, manganese, molybdenum, nickel and zinc. Despite the above improvements, still efforts are required to further optimize the productivity of the process and/or the quality (notably the purity) of the product. For instance, minimizing the pneumocandin C₀ impurity is the subject of US 2009/0291996 advocating to purify crude pneumocandin B₀ by chromatography followed by crystallization from a solvent-antisolvent mixture. Given the very high similarity between desired structure and impurity, not only in terms of the many different chemical reactive sites present in both molecules, but also in terms of charge, hydrophilicity and molecular weight, such a separation is laborious, time consuming and usually has a significant negative effect on the recovery yield of the desired product. Hence, there remains a need to further improve the pneumocandin B₀ fermentation process, either in terms of productivity of the desired product or in terms of reduction of undesired side products or both. Given the limited knowledge available on the mechanism of these highly complex natural processes combined with the endless permutations that could be envisaged in process development studies, such process improvements are difficult to predict and require inventive breakthroughs.

### DETAILED DESCRIPTION OF THE INVENTION

In the context of the present invention, the term “cyclopeptide” (also known as cyclic peptide or cyclic protein) refers to a polypeptide chain of which the amino and carboxyl termini are linked together with a peptide bond that forms a circular chain. The cyclopeptides of the present invention may be equipped with substituents such as acyl groups connected through an amide bond to an amino group. In addition the cyclopeptides of the present invention comprise natural and/or non-natural amino acids. In the context of the present invention the cyclopeptides comprise an amino acid chosen from the list consisting of 3-hydroxyproline, 3-hydroxy-4-methylproline, 4-hydroxyproline and proline. The number of amino acids linked together in the cyclopeptides of the present invention is from 3 to 20, preferably from 4 to 12, more preferably from 5 to 10, most preferably from 6 to 8. Preferred cyclopeptides in this respect are cyclohexapeptides such as aculeacin, echinocandin B (A30912A), FR901379, L-671329, mulundocandin, pneumocandin (pneumocandin A₀, A₁, A₂, A₃, A₄, A₅, A₆, B₀, B₁, B₂, B₅, B₆, C₀, D₀, D₂, E₀), S31794/F1 and sporiofungin. All such antifungals are structurally characterized by a cyclohexapeptide core, or nucleus, the amino group of one of the amino acids bearing a fatty acid acyl group forming a side chain.

The term “metal ion concentration” refers to the total amount of the metal ion referred to as added prior to or during the fermentation, per amount of initial medium present at the start of the fermentation and can be expressed in g.kg⁻¹, mg.kg⁻¹, mol.kg⁻¹, mmol.kg⁻¹ and the like.

The term “nutrient” refers to a chemical that an organism can use to live and grow or to a substance used in an organism's metabolism which must be taken in from its environment. Organic nutrients include carbohydrates, fats, proteins (or their building blocks, amino acids) and vitamins. Inorganic nutrients include water, oxygen and dietary minerals such as metal ions, examples of which are calcium, cobalt, copper, iron, magnesium, manganese, molybdenum, zinc and the like. A nutrient is said to be essential if it must be obtained from an external source, either because the organism cannot synthesize it or produces it in insufficient quantities. The effects of nutrients are dose-dependent and shortages are referred to as deficiencies.

In the first aspect of the present invention there is disclosed a method for the preparation of a cyclopeptide comprising an amino acid chosen from the list consisting of 3-hydroxyproline, 3-hydroxy-4-methylproline, 4-hydroxyproline and proline which method comprises fermenting a culture of Aspergillus sp., Coleophoma sp. or Glarea sp. in the presence of nutrients comprising calcium ions, copper ions, iron ions, magnesium ions, manganese ions, molybdenum ions and zinc ions, characterized in that the metal ion concentration of said calcium ions is at least 0.4 mg.kg⁻¹ and/or of said copper ions is at least 0.15 mg.kg⁻¹ and/or of said iron ions is at least 3 mg.kg⁻¹ and/or of said magnesium ions is at least 70 mg.kg⁻¹ and/or of said manganese ions is at least 5 mg.kg⁻¹ and/or of said molybdenum ions is at least 0.15 mg.kg⁻¹ and/or of said zinc ions is at least 0.7 mg.kg⁻¹.

Suitable microorganisms are Aspergillus sp., Coleophoma sp. and Glarea sp. Notably Aspergillus aculeatus (for instance for the production of aculeacin as described in Takeshima et al., J. Biochem. (1989) 105, 606), Aspergillus nidulans and Aspergillus rugulosus (for instance for the production of echinocandin B), Aspergillus sydowii (for instance for the production of mulundocandin as described in Mukhopadhyay et al., J. Antibiotics (1992) 45, 618), Coleophoma empetri F-11899 (for instance for the production of FR901379) or Glarea lozoyensis (for instance for the production of pneumocandins as described in WO 00/08197) are suitable in the context of the present invention. Optimal results in terms of productivity and decrease of side products are achieved when the cyclopeptide contains at least one amino acid chosen from the list consisting of 3-hydroxyproline, 3-hydroxy-4-methylproline, 4-hydroxyproline and proline. A preferred example is pneumocandin B₀ as produced by Glarea lozoyensis, preferably Glarea lozoyensis CBS 131548.

The predominant conclusion from the prior art such as WO 00/08197 is that fermentation of pneumocandin B₀ in Glarea lozoyensis benefits from a nutrient medium containing a high residual sugar concentration, trace elements, proline and threonine. The only investigation with respect to metals deals with the trace elements zinc, cobalt and nickel leading to the conclusion that zinc and cobalt reduce the titer of the main product with concomitant increase of the titer of unwanted impurities whereas nickel had no effect on the titer of the main product but increases the formation of almost all impurities. Similar detrimental effects are also reported for copper and zinc by N. Connors et al. (Appl. Microbiol. Biotechnol. (2000) 54, 814-818) and L. A. Petersen et al. (J. Ind. Microbiol. Biotechnol. (2001) 26, 216-221). Although there is no suggestion as to how to improve cyclopeptide fermentation, metal ion concentrations suggested in WO 00/08197, to be 0.23 mg.kg⁻¹ for calcium ions (0.83 mg.kg⁻¹ CaCl₂.2H₂O), 0.08 mg.kg⁻¹ for copper ions (0.21 mg.kg⁻¹ CuC1₂.2H₂O), 1.67 mg.kg⁻¹ for iron ions (8.3 mg.kg⁻¹ FeSO₄.7H₂O), 39.44 mg.kg⁻¹ for magnesium ions (0.4 g.kg⁻¹ MgSO₄.7H₂O), 2.70 mg.kg⁻¹ for manganese ions (8.3 mg.kg⁻¹ MnSO₄.H₂O), 0.09 mg.kg⁻¹ for molybdenum ions (0.16 mg.kg⁻¹ (NH₄)₆Mo₇O₂₄.4H₂O) and 0.39 mg.kg⁻¹ for zinc ions (1.7 mg.kg⁻¹ ZnSO₄.7H₂O).

In the present invention it is surprisingly found that both an increase in biomass growth, productivity and a decrease in unwanted impurities can be obtained when increasing the metal ion concentration of any or all of the metal ions calcium, copper, iron, magnesium, manganese, molybdenum and zinc to a level that is from 1.5 to 10 times as high as used in the prior art and against what is advocated in the prior art, preferably from 1.7 to 5 times as high, more preferably from 1.8 to 2.5 times as high. Thus, preferred metal ion concentrations for these metal ions according to the present invention are: calcium, between 0.4 and 1 mg.kg⁻¹; copper, between 0.15 and 0.5 mg.kg⁻¹; iron, between 3 and 10 mg.kg⁻¹; magnesium, between 70 and 200 mg.kg⁻¹; manganese, between 5 and 15 mg.kg⁻¹; molybdenum, between 0.15 and 0.5 mg.kg⁻¹;

zinc, between 0.7 and 2 mg.kg⁻¹. A major advantage of increased biomass growth and productivity is that fermentations can be performed under a carbon limitation regime. Any of the said seven metal ions may be present at the concentration ranges mentioned above, however also two or more or all seven metals may be present at said metal ion concentration ranges.

In one embodiment the cyclopeptide is isolated following its formation during fermentation. Isolation can be carried out according to procedures known to the skilled person such as precipitation, extraction using organic solvents, crystallization, chromatography and combinations thereof.

In another embodiment, for the production of pneumocandin B₀ a continuous or repeated discontinuous feed of nitrogen in the form of ammonia or ammonium hydroxide during the fermentation is found advantageous as high titers could be obtained while at the same time the consumption of proline could be drastically minimized. Proline is an amino acid that, according to WO 00/08197, is an important nutrient for reduction of the levels of pneumocandin C₀, however when following this prior art process also a high consumption of the relatively expensive proline occurs. Yet another advantage is that the present invention not only results in reduction of the amount of proline needed, but also in the circumvention of the need for proline dosing resulting in a less complicated and more robust process design. Preferably said feed of nitrogen in the form of ammonia or ammonium hydroxide during the fermentation is carried out such as to maintain an ammonia concentration in a range of from 0.1-5 g.L⁻¹.

In yet another embodiment, the cyclopeptide obtained according to the present invention may be further converted to other, semi synthetic cyclopeptides. Thus, pneumocandin B₀ is converted into caspofungin according to known procedures as described in WO 2010/128096 and references cited therein. This may be carried out with pneumocandin B₀ as directly obtained during fermentation but preferably with pneumocandin B₀ obtained during fermentation and further purified and/or isolated.

In the second aspect of the present invention there is disclosed a strain of a Glarea sp. that is Glarea lozoyensis CBS 131548, deposited at the Centraalbureau voor Schimmelcultures, Uppsalalaan 8, 3584 CT Utrecht, The Netherlands. The strain of the second aspect is suitable for producing cyclopeptides according to the method of the first aspect and thus exhibits increased productivity and/or decreased side product formation at increased metal ion concentrations, particularly upon production of pneumocandin B₀.

### LEGEND TO THE FIGURES

FIG. 1 depicts the production of pneumocandin B₀ by Glarea lozoyensis (see Examples for details). X-axis: time in hours after inoculation, Y-axis: production of pneumocandin B₀ in mg.kg⁻¹.

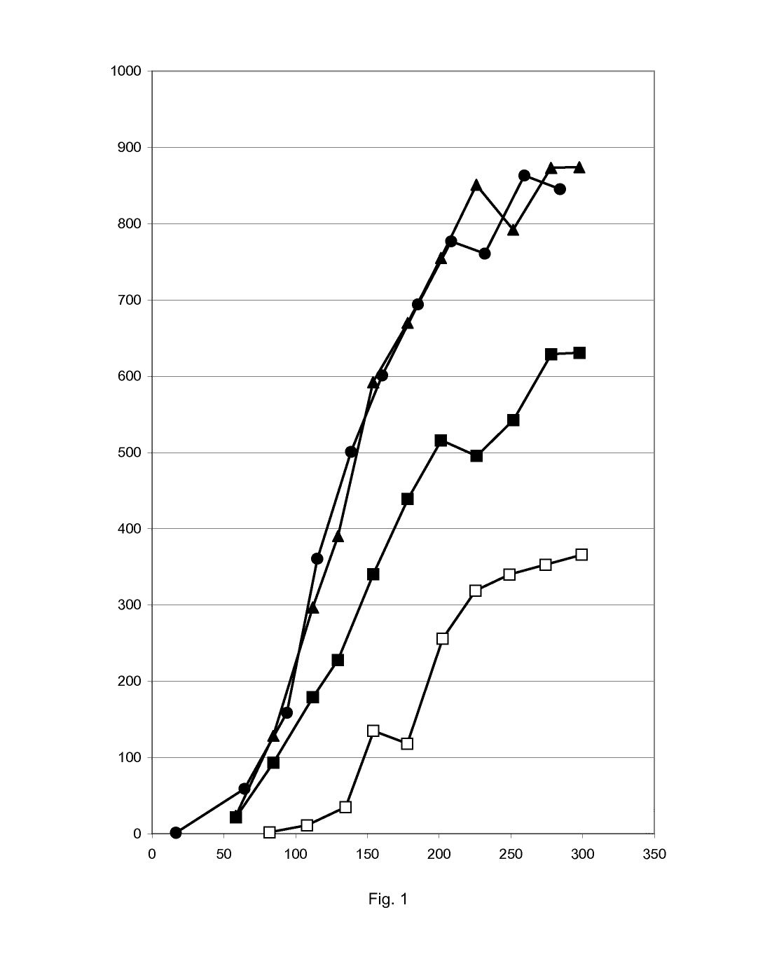

FIG. 2 depicts the ratio of the unwanted impurity pneumocandin C₀ during the production of pneumocandin B₀ by Glarea lozoyensis. X-axis: time in hours after inoculation, Y-axis: ratio (pneumocandin C₀/(pneumocandin B₀+pneumocandin C₀)).

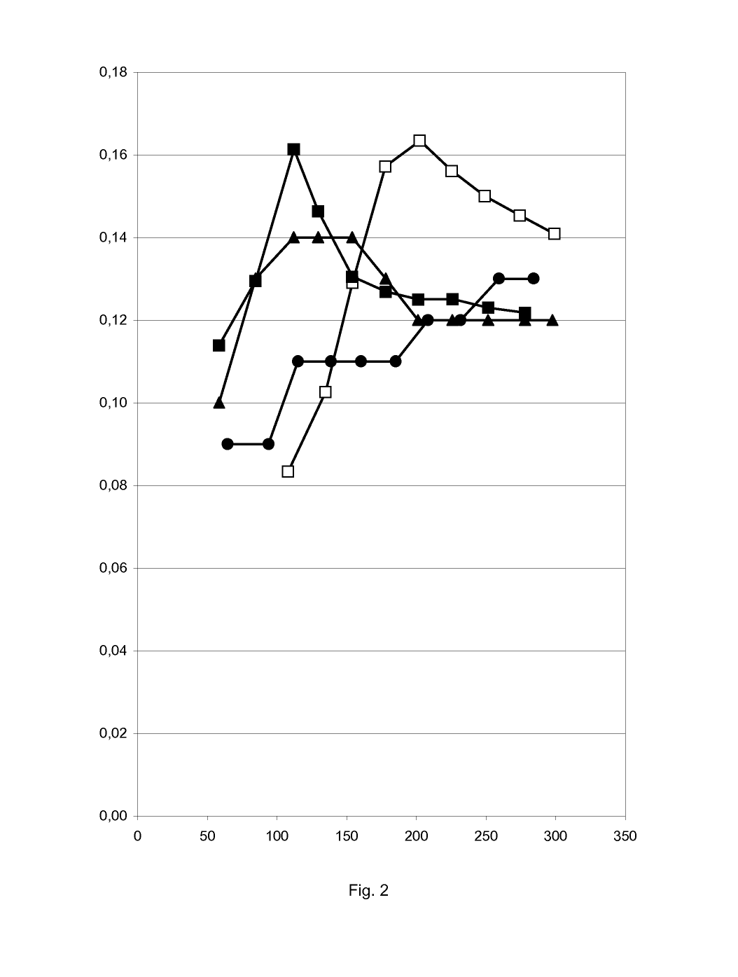

FIG. 3 depicts the biomass formation during the production of pneumocandin B₀ by Glarea lozoyensis. X-axis: time in hours after inoculation, Y-axis:

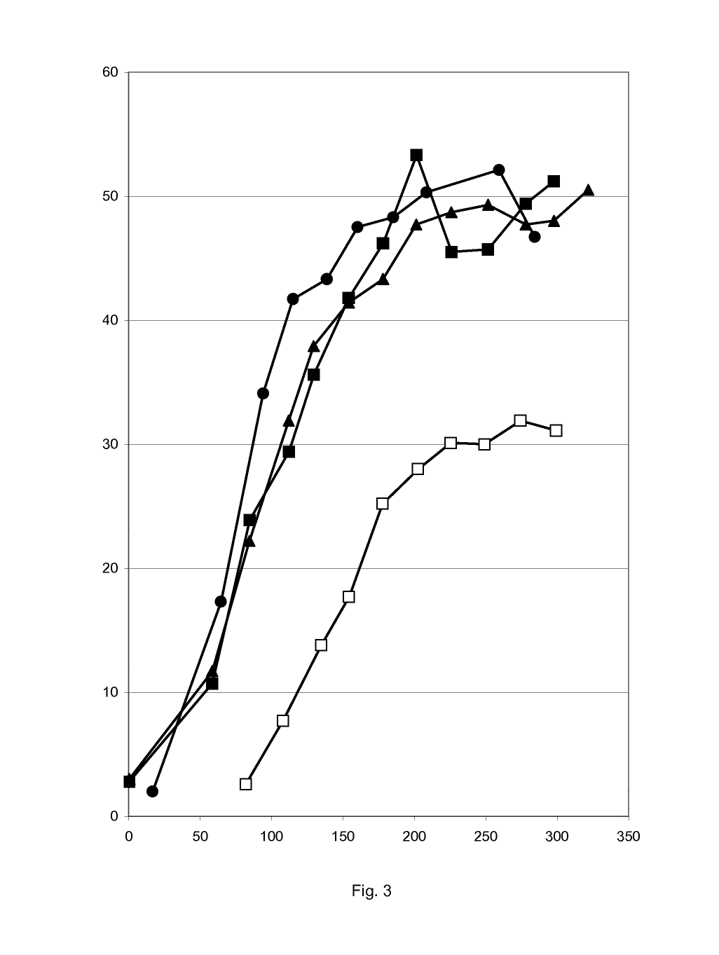

production of biomass in g.kg⁻¹.

FIG. 4 depicts the production of pneumocandin B₀ per gram of biomass by Glarea lozoyensis. X-axis: time in hours after inoculation, Y-axis: production of pneumocandin B₀ per gram of dry weight biomass in mg.g⁻¹.

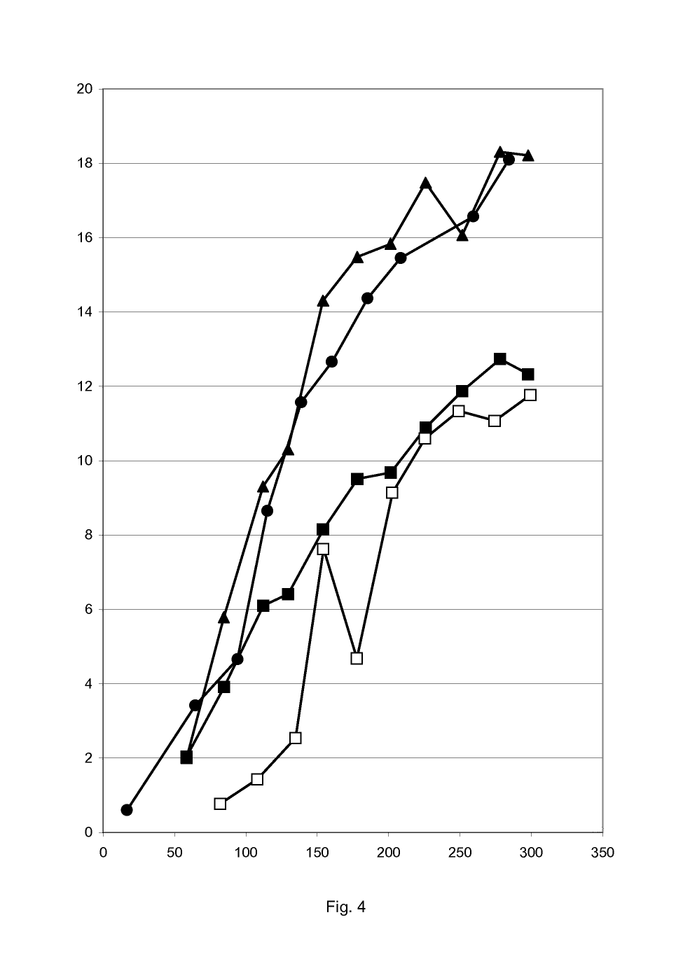

All Figures depict results in the four different media outlined below. In all media except medium 1, the fermentation is carried out under carbon limitation.
Medium 1 (□): Using Expresa yeast extract and metal ion concentrations of 0.23 mg.kg⁻¹ for Ca²⁺, 0.08 mg.kg⁻¹ for Cu²⁺, 1.67 mg.kg⁻¹ for Fe²⁺, 39.44 mg.kg⁻¹ for Mg²⁺, 2.70 mg.kg⁻¹ for Mn²⁺, 0.09 mg.kg⁻¹ for Mo⁶⁺ and 0.39 mg.kg⁻¹ for Zn²⁺.
Medium 2 (▪): As medium 1 with double metal ion concentrations.
Medium 3 (▴): As medium 1 with double metal ion concentrations and Gistex yeast extract paste LS instead of Expresa yeast extract.
Medium 4 (): As medium 1 with triple metal ion concentrations and Gistex yeast extract paste LS instead of Expresa yeast extract.

### EXAMPLES

### Equipment Used

Laboratory fermentors with a gross volume of 10 L were equipped with a Rushton turbine and a pH and DO probe both of Ingold. Control of fermentations took place with

Braun DCCU-2 equipment and a Braun MFCS/WIN system version 3.0 both supplied by B. Braun Biotech International. Shake flasks (2000 mL) equipped with three bottom baffles and a foam plug were used in an Innova 4330 refrigerated incubation shaker.

### Stock Solutions

### Glucose Stock Solution:

Dextrose (500 g) was dissolved or suspended in approximately 800 mL of de-mineralized water, the volume was adjusted to 1 L and the mixture was sterilized for 20 minutes at 121° C. and cooled down to room temperature before use.

### Trace Elements Stock Solution:

| | | Concentration |
|---|---|---|
| Component | Formula | (g · kg⁻¹) |
| Hydrochloric acid 4N | HCl | 250 |
| Iron(II)sulphate.7aq | FeSO₄•7H₂O | 8.3 |
| Manganese sulphate.1aq | MnSO₄•H₂O | 8.3 |
| Copper chloride.2aq | CuCl₂•2H₂O | 0.21 |
| Calcium chloride.2aq | CaCl₂•2H₂O | 0.83 |
| Boric acid | H₃BO₃ | 0.47 |
| Ammonium molybdate.4aq | (NH₄)₆Mo₇O₂₄•4H₂O | 0.16 |
| Zinc sulphate.7aq | ZnSO₄•7H₂O | 1.7 |

The indicated amount of hydrochloric acid was added to approximately 250 mL of de-mineralized water after which all other components were dissolved and the weight was adjusted to 1 kg by addition of water. The mixture was stored at 4° C. (shelf life 6 months).

### Seed Fermentation

Expresa 2200S Yeast Extract Medium Preparation (Seed medium 1):

| | | Concentration | |
|---|---|---|---|
| Component | Formula | (g · L⁻¹) | Fraction |
| Citric acid monohydrate | C₆H₈O₇•H₂O | 5 | 1 |
| Expresa 2200S yeast extract | N.A. | 8.5 | 1 |
| Di-potassium hydrogen | K₂HPO₄ | 2.24 | 1 |
| phosphate | | | |
| Magnesium sulphate | MgSO₄•7H₂O | 0.4 | 1 |
| Sodium sulphate | Na₂SO₄ | 5 | 1 |
| Trace elements stock solution | N.A. | 1.0 | 1 |
| 50% Dextrose stock solution | C₆H₁₂O₆•H₂O | 25 mL | 2 |

Gistex Yeast Extract LS Paste Medium Preparation (Seed medium 2):

| | | Concentration | |
|---|---|---|---|
| Component | Formula | (g · L⁻¹) | Fraction |
| Citric acid monohydrate | C₆H₈O₇•H₂O | 5 | 1 |
| Gistex yeast extract LS paste | N.A. | 12 | 1 |
| Di-potassium hydrogen | K₂HPO₄ | 2.24 | 1 |
| phosphate | | | |
| Magnesium sulphate | MgSO₄•7H₂O | 0.4 | 1 |
| Sodium sulphate | Na₂SO₄ | 5 | 1 |
| Trace elements stock solution | N.A. | 1.0 | 1 |
| 50% Dextrose stock solution | C₆H₁₂O₆•H₂O | 25 mL | 2 |

Materials of fraction 1 were dissolved or suspended in the order given, in 80% of the final volume of tap water. The pH was brought to 5.7±0.1 with NaOH 4N or H₂SO₄ 4N and the volume was adjusted to the desired volume. Shake flasks (2000 mL) equipped with three baffles were filled with 500 mL of medium and were sterilized for 20 minutes at 121° C. After sterilization and cooling down 25 mL of sterile dextrose solution was added to the 2000 mL flasks containing fraction 1.

### Seed Incubation:

| | Phase 1 | Phase 2 |
|---|---|---|
| Seed¹⁾ | Complete vial | 0.5 ml of phase 1 |
| | (approximately 1 mL) | |
| Culture volume | 500 ml | 500 ml |
| Flask gross volume | 2000 ml | 2000 ml |
| Type of shake flask | With baffles | With baffles |
| Type of plug | Foam plug | Foam plug |
| Temperature incubator | 25.0° C. | 25.0° C. |
| Agitation speed/pitch | 220 rpm/2.54 cm | 220 rpm/2.54 cm |
| Incubation time | 168 ± 4 hours | 216 ± 4 hours |
| Transfer criterion | Time | Time |

¹⁾Glarea lozoyensis CBS 131548, deposited at the Centraalbureau voor Schimmelcultures, Utrecht, The Netherlands. Vials comprise mycelium in glycerol solution

In the next stage, the fermentor was inoculated with the whole content of seed phase 2.

### Fermentation

For the fermentation of pneumocandin B₀, four different media were used:

### Medium 1:

Based on WO 00/08197, Table 2; concentrations of di-potassium hydrogen phosphate, magnesium sulphate, trace elements stock solution and L-proline were equal. Sodium sulphate was added to increase osmotic pressure. The original recipe contained mono sodium glutamate, which was replaced on N-basis replaced by ammonium sulphate and Difco Yeast extract, which was on N-basis replaced by Expresa yeast extract. Citric acid was added as a chelating agent to prevent precipitations.

### Medium 2:

As medium 1 however with double concentrations of di-potassium hydrogen phosphate, magnesium sulphate and trace elements stock solution.

### Medium 3:

As medium 1 however with double concentrations of di-potassium hydrogen phosphate, magnesium sulphate and trace elements stock solution and with Gistex yeast extract LS paste instead of Expresa yeast extract.

### Medium 4:

As medium 1 however with triple concentrations of di-potassium hydrogen phosphate, magnesium sulphate and trace elements stock solution and with Gistex yeast extract LS paste instead of Expresa yeast extract. Also the fermentations using this medium were carried out with a simplified feed scheme in which the sugar, normally dosed during the exponential part of the feed, was now dosed directly in the batch.

| | Medium | | | | | |
|---|---|---|---|---|---|---|
| | | 1 | 2 | 3 | 4 | |
| Component | Formula | g · kg⁻¹ | g · kg⁻¹ | g · kg⁻¹ | g · kg⁻¹ | Fraction |
| Citric acid monohydrate | C₆H₈O₇•H₂O | 5.0 | 10.0 | 10.0 | 5.0 | 1 |
| Ammonium sulfate | (NH₄)₂SO₄ | 7.0 | 7.0 | 7.0 | 7.0 | 1 |
| Yeast extract powder | Expresa 2200S | 8.5 | 8.5 | | — | 1 |
| Yeast extract paste LS | DSM | — | — | 12.0 | 12.0 | 1 |
| Di potassium phosphate | K₂HPO₄ | 2.24 | 4.48 | 4.48 | 6.72 | 1 |
| Magnesium sulphate | MgSO₄•7H₂O | 0.4 | 0.8 | 0.8 | 1.2 | 1 |
| L-proline | C₅H₉NO₂ | 20.0 | 20.0 | 20.0 | 20.0 | 1 |
| Sodium sulfate | Na₂SO₄ | 5.0 | 5.0 | 5.0 | 5.0 | 1 |
| Trace elements stock solution | Not applicable | 1.0 | 2.0 | 2.0 | 3.0 | 1 |
| Anti foam Basildon 86/013 | Not applicable | 0.05 | 0.05 | 0.05 | 0.05 | 1 |
| D-Fructose | C₆H₁₂O₆ | 40 | 40 | 40 | 70 | 2 |

Media were prepared in two fractions that are sterilized separately. Fraction 1 is ⅚ of the total volume after sterilization, fraction 2 is ⅙ of the total volume.

Preparation fraction 1: components were dissolved in tap water, representing 80% of the fraction volume, after which water was added to the appropriate volume and the solution was sterilized for 30 minutes at 121° C.

Preparation fraction 2: D-fructose was dissolved in tap water, representing 50% of the fraction volume, after which water was added to the appropriate volume and the solution was sterilized for 30 minutes at 121° C.

Both fractions were added to the fermentor after cooling down and the pH was adjusted to 5.35±0.15 using ammonium hydroxide 12.5%.

### Fermentation Conditions:

The fermentation was carried out under atmospheric pressure using an amount of seed of 8.3%. The DO probe was calibrated at 100% under the fermentation conditions described below, and before inoculation. During the fermentation the following set points were applied:

| Item | Set point | Remarks |
|---|---|---|
| Temperature (° C.) | 25.0 ± 0.1 | |
| Airflow (VVM) | 1 | |
| pH | 5.35 ± 0.15 | Control with H2SO4 4N and NaOH |
| | | 4N or NH4OH (Depending on |
| | | the experiment) |
| Stirrer speed (rpm) | 150 | Maximum 900 rpm see also |
| | Cascade DO | DO-control |
| DO-control (scale | 75 ± 5% | Calibrate the DO- electrode at 100 |
| units) | | scale units Primary control with |
| | | stirrer speed (automatically) |
| Anti foam | If needed | Anti foam Basildon 86/013 |
| End of fermentation | 240-288 h | |

### Feed Preparation:

The feed (D-fructose 200 g.kg⁻¹) was prepared by dissolving D-fructose in 50% of the desired volume of hot tap water after which water was added to the desired value and the solution was sterilized for 20 minutes at 121° C.

### Feed Start:

After start of growth the pH drops to the lower boundary of the control (pH=5.20) and base was added. When batch sugar was finished the pH value increased. Feed was started when the pH reached a value of 5.35.

### Feed Profile:

For fermentations 1-3 using 40 g.kg ⁻¹ D-fructose as batch dosage the following feed profile was applied:

| Time | Set point |
|---|---|
| (Hours after feed start) | (g_S · kg⁻¹ starting weight) |
| FS - 36 | 3.0 × e^{(0.015×HAFS)} |
| 36 EOF | 5.15 |

For fermentation 4 with medium 4 the sugar (70 g.kg⁻¹ D-fructose), normally dosed during the exponential part of the profile, was already added to the batch. For this experiment the following feed profile was used:

| Time | Set point |
|---|---|
| (Hours after feed start) | (g_S · kg⁻¹ starting weight) |
| 36 EOF | 5.15 |

1. Method for the preparation of a cyclopeptide comprising an amino acid chosen from the list consisting of 3-hydroxyproline, 3-hydroxy-4-methylproline, 4-hydroxyproline and proline which method comprises fermenting a culture of Aspergillus sp., Coleophoma sp. or Glarea sp. in the presence of nutrients comprising calcium ions, copper ions, iron ions, magnesium ions, manganese ions, molybdenum ions and zinc ions, characterized in that the metal ion concentration of said calcium ions is between 0.4 and 1 mg.kg⁻¹ and/or of said copper ions is between 0.15 and 0.5 mg.kg⁻¹ and/or of said iron ions is between 3 and 10 mg.kg⁻¹ and/or of said magnesium ions is between 70 and 200 mg.kg⁻¹ and/or of said manganese ions is between 5 and 15 mg.kg⁻¹ and/or of said molybdenum ions is between 0.15 and 0.5 mg.kg⁻¹ and/or of said zinc ions is between 0.7 and 2 mg.kg⁻¹.

2. Method according to claim 1 wherein said calcium ions, copper ions, iron ions, magnesium ions, manganese ions, molybdenum ions and zinc ions are all present at said metal ion concentrations.

3. Method according to claim 1 wherein said culture is Aspergillus aculeatus, Aspergillus nidulans, Aspergillus rugulosus, Aspergillus sydowii, Coleophoma empetri F-11899 or Glarea lozoyensis.

4. Method according to claim 1 wherein said cyclopeptide is aculeacin, echinocandin B, FR901379, L-671329, mulundocandin, pneumocandin A₀, pneumocandin A₁, pneumocandin A₂, pneumocandin A₃, pneumocandin A₄, pneumocandin A₅, pneumocandin A₆, pneumocandin B₀, pneumocandin B₁, pneumocandin B₂, pneumocandin B₅, pneumocandin B₆, pneumocandin C₀, pneumocandin D₀, pneumocandin D₂, pneumocandin E₀, S31794/F1 or sporiofungin.

5. Method according to claim 1 wherein said cyclopeptide is pneumocandin B₀ and said culture is Glarea lozoyensis.

6. Method according to claim 1 subsequently comprising isolating said cyclopeptide.

7. Method according to claim 1 wherein said cyclopeptide is pneumocandin B₀ further comprising conversion of said pneumocandin B₀ into caspofungin or a pharmaceutically acceptable salt thereof.

8. Method according to claim 1 wherein ammonia and/or ammonium hydroxide is added during said fermenting in such a way as to maintain the ammonia concentration in a range of from 0.1-5 g.L⁻¹.

9. A microorganism that is Glarea lozoyensis CBS 131548.
